(19) United States
(12) Patent Application Publication
(10) Pub. No.: US 2005/0203023 A1
(43) Pub. Date: Sep. 15, 2005

(54) Use of rAFP inhibit or prevent apoptosis

(76) Inventors: Robert A. Murgita, Montreal (CA); Robert Mulroy, Cambridge, MA (US); Stace Lindsay, Cambridge, MA (US)

Correspondence Address: CLARK & ELBING LLP, 101 FEDERAL STREET, BOSTON, MA, 02110, US

(21) Appl. No.: 11/120,753

(22) Filed: May 2, 2005

Related U.S. Application Data
(63) Continuation of application No. 10/069,623, filed on Dec. 16, 2002, now abandoned.
(60) Provisional application No. 60/152,166, filed on Sep. 2, 1999.

(51) Int. Cl. A61K 38/17

(52) U.S. Cl. 514/12

(57) ABSTRACT

A method of inhibiting apoptosis in a cell by administering to the cell an apoptosis inhibiting amount of recombinant human alpha-feta protein or an apoptosis-inhibiting fragment thereof.

### BACKGROUND OF THE INVENTION

The invention related to methods of inhibiting apoptosis.

There are two general ways in which cells die. The most easily recognized way is by necrosis, which is usually caused by an injury that is severe enough to disrupt cellular homeostasis. Typically, the cell's osmotic pressure is disturbed and, consequently, the cell swells and then ruptures. When the cellular contents are spilled into the surrounding tissue space, an inflammatory response often ensues.

The second general way by which cells die is referred to as apoptosis, or programmed cell death. Apoptosis often occurs so rapidly that it is difficult to detect. This may help to explain why the involvement of apoptosis in a wide spectrum of biological processes has only recently been recognized.

The apoptosis pathway has been highly conserved throughout evolution, and plays a critical role in embryonic development, viral pathogenesis, cancer, autoimmune disorders, and neurodegenerative disease. For example, inappropriate apoptosis may cause or contribute to AIDS, Alzheimer's Disease, Parkinson's Disease, Amyotrophic Lateral Sclerosis (ALS), retinitis pigmentosa and other diseases of the retina, myelodysplastic syndrome (e.g. aplastic anemia), toxin-induced liver disease, including alcoholism, and ischemic injury (e.g. myocardial infarction, stroke, and reperfusion injury). Conversely, the failure of an apoptosis response has been implicated in the development of cancer, particularly follicular lymphoma, p53-mediated carcinomas, and hormone-dependent tumors, in autoimmune disorders, such as lupus erythematosis and multiple sclerosis, and in viral infections, including those associated with herpes virus, poxvirus, and adenovirus.

In patients infected with HIV-1, mature CD4⁺T lymphocytes respond to stimulation from mitogens or super-antigens by undergoing apoptosis. However, the great majority of these cells are not infected with the virus. Thus, inappropriate antigen-induced apoptosis could be responsible for the destruction of this vital part of the immune system in early stages of HIV infection.

### SUMMARY OF THE INVENTION

In general, the invention features the inhibition of apoptosis in a cell, e.g., a cell in a mammal such as a human patient, by contacting the cell with recombinant alpha-fetaprotein (“rHuAFP”) or an effective fragment thereof, or with nucleic acid encoding rHuAFP. The invention, in inhibiting apoptosis, can provide therapy for diseases in which inappropriate apoptosis is a feature, including AIDS or HIV infection, neurodegenerative diseases such as ALS, a myelodysplastic syndrome, or an ischemic injury such as occurs in stroke, myocardial infarction, reperfusion injury, or a toxin-induced liver disease. Other features and advantages of the invention will be apparent from the detailed description of the invention, the drawings, and the claims.

### BRIEF SUMMARY OF THE DRAWINGS

FIG. 1 is the nucleotide sequence (SEQ ID NO: 1) and deduced amino acid sequence (SEQ ID NO: 2) of the cDNA encoding human alpha-fetoprotein, and the amino acid sequences (SEQ ID NOs: 3-8) of rHuAFP fragments.

FIG. 2 is the SDS-PAGE analysis of rHuAFP Fragment I (SEQ ID NO: 8) (Lane A, MW marker; Lane B, native human alpha-fetoprotein (AFP); Lane C, unpurified rAFP; Lane D, rAFP Fragment I, and Lane E, AFP (amino acids 1-590 of FIG. 1, SEQ ID NO: 2).

### DETAILED DESCRIPTION OF THE INVENTION

Production of Recombinant Human Alpha-fetoprotein

Recombinant AFP can be produced in any standard recombinant protein production system, including prokaryotic cells such as E. coli, and eukaryotic systems such as yeast, mammalian (e.g., CHO cells) and insect cells. Prokaryotic production of rHuAFP is described in Murgita U.S. Pat. No. 5,384,250, hereby incorporated by reference.

The methods of the invention can also employ biologically active fragments of rHuAFP. A biologically active fragment of rHuAFP is one that possesses at least one of the following activities: (a) directs a specific interaction with a target cell, e.g., binds to a cell expressing a receptor that is recognized by rHuAFP (e.g., the membrane of a cancer cell such as MCF-7); or (b) halts, reduces, or inhibits apoptosis (e.g., binds to a cell surface receptor and imparts an anti-apoptosis signal). The ability of rHuAFP fragments to bind to a receptor which is recognized by rHuAFP can be tested using any standard binding assay known in the art.

In general, fragments of rHuAFP are produced according to the techniques of polypeptide expression and purification described in U.S. Pat. No. 5,384,250. DNA sequences encoding fragments of rHuAFP can be generated by standard techniques and cloned into expression vectors for expression in recombinant cells. Expressed fragments can be isolated by various chromatographic and/or immunological methods known in the art. Lysis and fractionation of rHuAFP-containing cells prior to affinity chromatography may be performed by standard methods. Once isolated, the recombinant protein can, if desired, be further purified, e.g., by high performance liquid chromatography (see, e.g., Fisher, Laboratory Techniques In Biochemistry and Molecular Biology, Work and Burdon, eds., Elsevier, 1980).

Recombinant HuAFP fragments can be assayed by immunological procedures, such as Western blot, immunoprecipitation analysis of recombinant cell extracts, or immunofluorescence (using, e.g., the methods descrbed in Ausubel et al., Current Protocols In Molecular Biology, Greene Publishing Associates and Wiley Interscience (John Wiley & Sons), New York, 1994).

Useful rHuAFP fragments preferably have at least 20 contiguous amino acids, preferably at least 50 contiguous amino acids, more preferably at least 100 contiguous amino acids, and most preferably at least 200 to 400 or more contiguous amino acids in length.

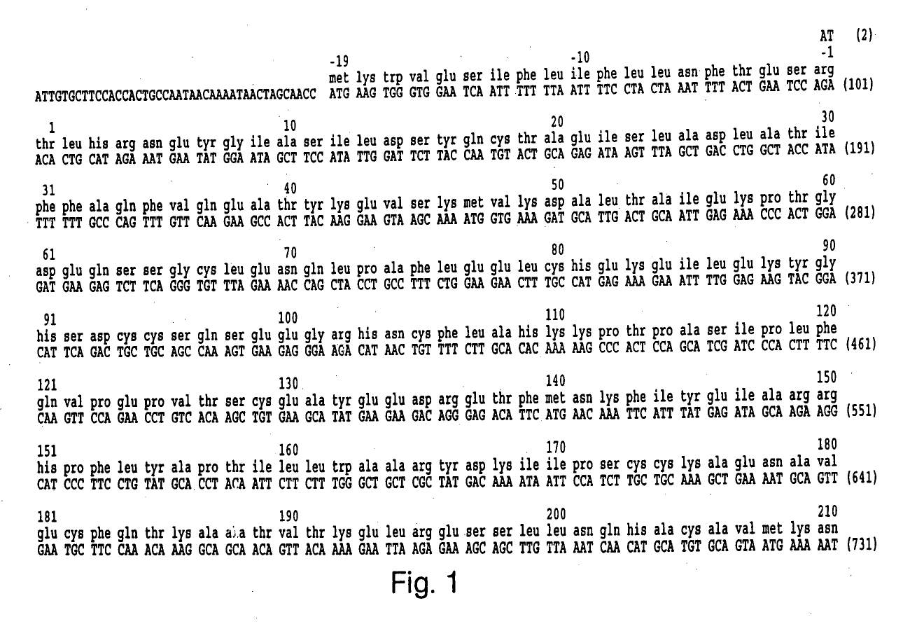

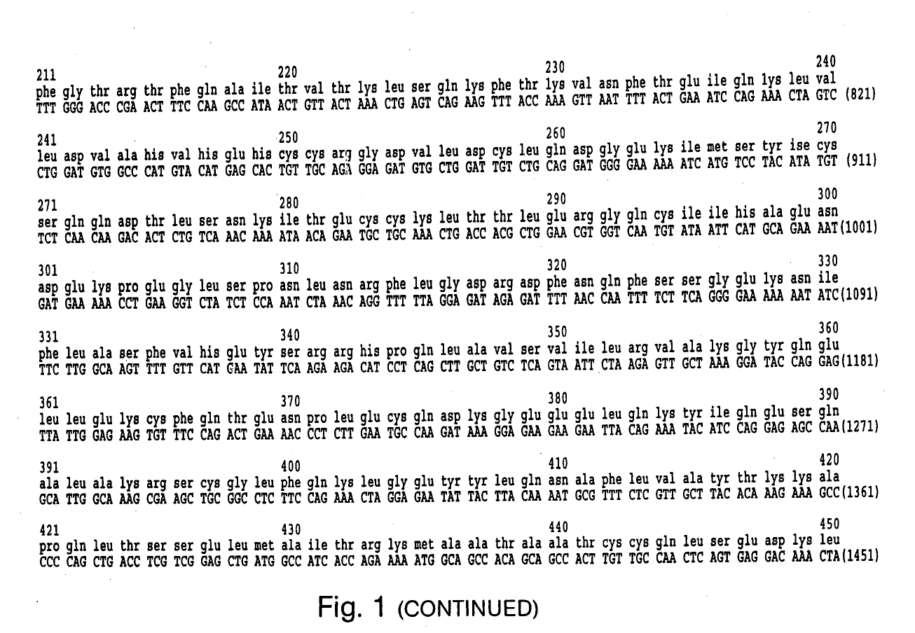

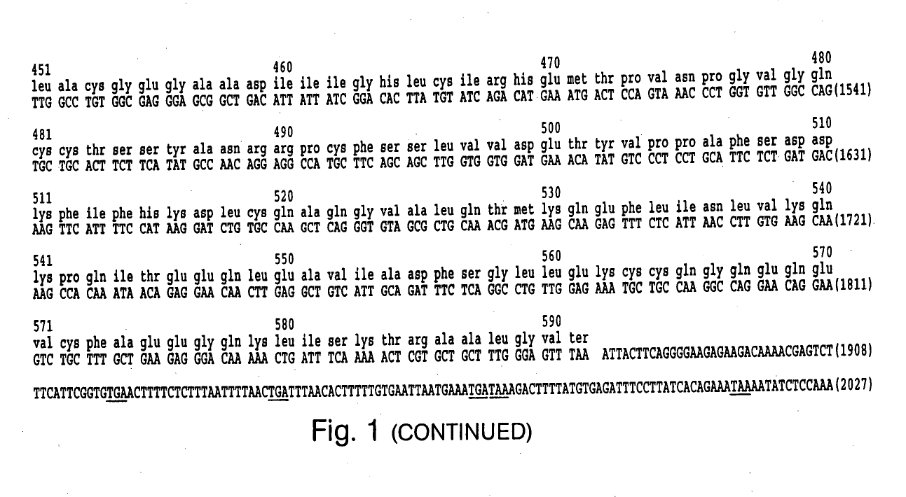

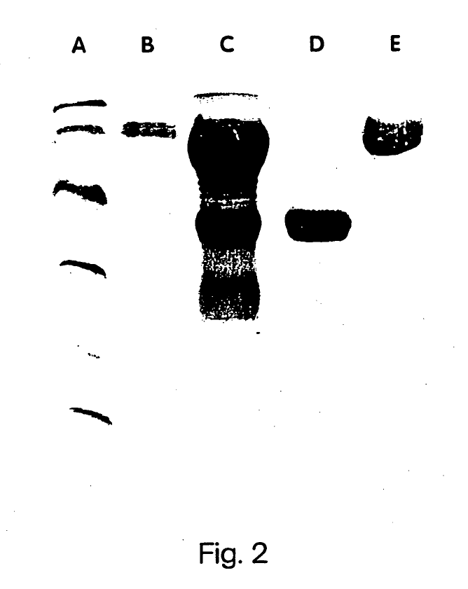

Recombinant HuAFP fragments of interest include, but are not limited to, Domain I (amino acids 1 (Thr)-197 (Ser), see FIG. 1, SEQ ID NO: 3), Domain II (amino acids 198(Ser)-389 (Ser), see FIG. 1, SEQ ID NO: 4), Domain III (amino acids 390 (Gln)-590 (Val), see FIG. 1, SEQ ID NO: 5), Domain I+II (amino acids 1 (Thr)-389 (Ser), see FIG. 1, SEQ ID NO: 6), Domain II+III (amino acids 198 (Ser)-590 (Val), see FIG. 1, SEQ ID NO: 7), and rHuAFP Fragment I (amino acids 266 (Met)-590 (Val), see FIG. 1, SEQ ID NO: 8).

By “inhibiting apoptosis” is meant a decrease in the number of cells which undergo apoptosis relative to an untreated control. Preferably, the decrease is at least 25%, more preferably the decrease is 50%, and most preferably the decrease is at least one-fold.

Apoptosis Assays

Apoptosis assays are described in the following references. Assays for apoptosis in lymphocytes are disclosed by, for example: Li et al., “Induction of apoptosis in uninfected lymphocytes by HIV-1 Tat protein”, Science 268:429-431, 1995; Gibellini et al., “Tat-expressing Jurkat cells show an increased resistance to different apoptosis stimuli, including acute human immunodeficiency virus-type 1 (HIV-1) infection:, Br. J. Haematol. 89:24-33, 1995; Martin et al., “HIV-1 infection of human CD4⁺ T cells in vitro. Differential induction of apoptosis in these cells.” J. Immunol. 152:330-42, 1994; Terai et al., “Apoptosis as a mechanism o cell death in cultured T lymphoblasts acutely infected with HIV-1”, J. Clin Invest. 87:1710-5, 1991; Dhein et al., “Autocrine T-cell suicide mediated by APO-1/(Fas/CD95) 11, Nature 373:438-441, 1995; Katsikis et al., “Fas antigent stimulation induces marked apoptosis of T lymphocytes in human immunodeficiency virus-infected individuals”, J. Exp. Med. 1815:2029-2036, 1995; Estendorp et al., “Sensitization of T cells to CD95-mediated apoptosis by HIV-1 Tat and gp120”, Nature 375:497, 1995; DeRossi et al., Virology 198:234-44, 1994.

Assays for apoptosis in fibroblasts are disclosed by, for example:Vossbeck et al;, “Direct transforming activity of TGF-beta on rat fibroblasts,” Int. J. Cancer 61:92-97, 1995; Goruppi et al., “Dissection of c-myc domains involved in S phase induction of NIH3T3 fibroblasts,” Oncogene 9:1537-44, 1994; Fernandez et al., “Differential sensitivity of normal and Ha-ras transformed C3H mouse embryo fibroblasts to tumor necrosis factor: induction of bcl-2, c-myc, and manganese superoxide dismutase in resistant cells,” Oncogene 9:2009-17, 1994; Harrington et al., “c-Myc-induced apoptosis in fibroblasts is inhibited by specific cytokines,” EMBO J., 13:3286-3295, 1994; Itoh et al., “A novel protein domain required for apoptosis. Mutational analysis of human Fas antigen,” J. Biol. Chem. 268:10932-7, 1993.

Assays for apoptosis in neuronal cells are disclosed by, for example:Melino et al., “Tissue transglutaminase and apoptosis: sense and antisense transfection studies with human neuroblastoma cells,” Mol. Cell Biol. 14:6584-6596, 1994; Rosenbaum et al., “evidence for hypoxiainduced, programmed cell death of cultured neurons,” Ann. Neurol. 36:864-870, 1994; Sato et al., “Neuronal differentation of PC12 cells as a result of prevention of cell death by bcl-2,” J. Neurobiol 25:1227-1234, 1994; Ferrari et al., “N-acetylcysteine D- and L-stereoisimers prevents apoptosis death of neuronal cells,” J. Neurosci. 1516:2857-2866, 1995; Talley et al., “Tumor necrosis factor alpha-induced apoptosis in human neuronal cells: protection by the antioxidant N-acetylcysteine and the genes bcl-2 and crma,” Mol. Cell Biol. 1585:2359-2366, 1995; Talley et al., “Tumor Necrosis Factor Alpha-Induced Apoptosis in Human Neuronal Cells: Protection by the Antioxidant NAcetylcysteine and the Genes bcl-2 and crmA,” Mol. Cell. Biol. 15:2359-2366, 1995; and Walkinshaw et al., “Induction of apoptosis in catecholaminergic PC12 cells by L-DOPA. Implication for the treatment of Parkinson's disease,” J. Clin. Invest. 95:2458-2464, 1995.

Assays for apoptosis in insect cells are disclosed by, for example: Clem et al., “Prevention of apoptosis by a baculovirus gene during infection of insect cells,” Science 254:1388-90, 1991; Crook et al., “An apoptosis-inhibiting baculovirus gene with a zince finger-like motif,” J. Virol. 67:2168-74, 1993; Rabizadeh et al., “Expression of the baculovirus p35 gene inhibits mammalian neural cell death,” J. Neurochem. 61:2318-21, 1993; Birnbaum et al., “an apoptosis inhibiting gene from a nuclear polyhedrosis virus encoding a polypeptide with Cys/His sequence motifs,” J. Virol. 68:2521-8, 1994; and Clem et al., “Control of programmed cell death by the baculovirus genes p35 and IAP,” Mol. Cell. Biol. 14:5212-5222, 1994.

Gene Therapy

rHuAFP-encoding genes can be used according to the invention in anti-apoptosis gene therapy. In particular, a functional rHuAFP gene may be used to sustain neuronal cells that undergo apoptosis in the course of a neurodegenerative disease; lymphocytes (i.e., T cells and B cells); or cells that have been injured by ischemia.

Retroviral vectors, adenoviral vectors, adeno-associated viral vectors, or other viral vectors with the appropriate tropism for cells likely to be involved in apoptosis (for example, epithelial cells) may be used as a gene transfer delivery system for a therapeutic rHuAFP gene construct. Numerous vectors useful for this purpose are known (Miller, Human Gene Therapy 15-15, 1990; Friedman, Science 244:1275-1281, 1989; Eglitis and Anderson, BioTechniques 6:608-614, 1988; Tolstoshev and Anderson, Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechniques 7:980-990, 1989; Le Gal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346). Non-viral approaches may also be employed for the introduction of therapeutic DNA into cells otherwise predicted to undergo apoptosis. For example rHuAFP may be introduced into a neuron or a T cell by lipofection (Felgner et al., Proc. Natl. Acad. Sci. USA 84:7413, 1987; Ono et al., Neurosci. Lett. 117:259, 1990; Brigham et al., Am. J. Med. Sci. 298:278, 1989; Staubinger et al., Meth. Enz. 101:512, 1983), asialorosonucoid-polylysine conjugation (Wu et al., J. Biol. Chem. 263:14621, 1988; Wu et al., J. Biol. Chem. 264:16985, 1989); or, less preferably, microinjection under surgical conditions (Wolff et al., Science 247:1465, 1990).

For any of the methods described above, the therapeutic rHuAFP DNA construct is preferably applied to the site of the predicted apoptosis event (for example, by injection), or to tissue in the vicinity of the predicted apoptosis event, or to a blood vessel supplying the cells predicted to undergo apoptosis.

rHuAFP expression can be directed from any suitable promoter (e.g., the human cytomegalovirus (CMV), simian virus 40 (SV40), or metallothionein promoters), and regulated by any appropriate mammalian regulatory element. For example, if desired, enhancers that preferentially direct gene expression in neural cells, T cells, or B cells may be used to direct rHuAFP expression. Alternatively, if an rHuAFP genomic clone is used in a therapeutic construct, regulation may be mediated by the cognate regulatory sequences or, if desired, by regulatory sequences derived from a heterologous source, including any of the promoters or regulatory elements described above.

Alternatively, rHuAFP gene therapy is accomplished by direct adminstration of the rHuAFP mRNA or antisense rHuAFP mRNA to a cell that is expected to undergo apoptosis. The mRNA may be produced and isolated by any standard technique, but is most readily produced by in vitro transcription using an rHuAFP cDNA under the control of a high efficiency promoter (e.g., the T7 promoter). Administration of rHuAFP mRNA to cells can be carried out by any of the methods for direct nucleic acid adminstration described below.

Ideally, the production of rAFP protein by any gene therapy approach will result in cellular levels of rAFP that are at least equivalent to the normal, cellular level of rHuAFP in an unaffected cell. Treatment by any rHuAFP-mediated gene therapy approach may be combined with more traditional therapies.

Administration of rAFP Polypeptides

Another therapeutic approach of the invention involves adminstration of recombinant rHuAFP, either directly to the site of a predicted apoptosis event (for example, by injection) or systemically (for example, by any conventional recombinant protein adminstration technique). The dosage of rHuAFP depends on a number of factors, including the size and health of the individual patient, but, generally, between 0.1 mg and 100 mg are administered per day to an adult in a pharmaceutically-acceptable formulation. Administration may begin before or after the patient is symptomatic. Any appropriate route of adminstration may be employed, for example, administration may be parenteral, intravenous, intraarterial, subcutaneous, intramuscular, intracranial, intraorbital, ophthalmic, intraventricular, intracapsular, intraspinal, intracisternal, intraperitoneal, intranasal, aerosol, or oral. Therapeutic formulations may be in the form of liquid solutions or suspensions; for oral administration, formulations may be in the form of tablets or capsules; and for intranasal formulations, in the form of powers, nasals drops, or aerosols.

Methods well known in the art of making formulations are found, for example, in Remington's Pharmaceutical Sciences, (18^{th} edition), ed. A. Gennaro, 1990, Mack Publishing Company, Easton, Pa. Formulations for parenteral adminstration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of rHuAFP.

Treatment with an rHuAFP protein or gene may be combined with more traditional therapies for the disease such as surgery, steroid therapy, or chemotherapy for autoimmune disease; antiviral therapy for AIDS; and tissue plasminogen activator (TPA) for ischemic injury.

Other Embodiments

All publications and patent applications mentioned in this specification are herein incorporated by reference to the same extent as if each. independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features hereinbefore set forth, and follows in the scope of the appended claims.

1. A method of inhibiting apoptosis in a cell, said method comprising administering to said cell an apoptosis inhibiting amount of rHuAFP or an apoptosis-inhibiting fragment thereof.

2. The method of claim 1, wherein said cell is in a mammal.

3. The method of claim 2, wherein said mammal is human.

4. The method of claim 3, wherein said human is infected with HIV, or has a neurodegenerative disease, a myelodysplastic syndrome, or an ischemic injury.

5. The method of claim 4, wherein said ischemic injury is caused by a myocardial infarction, a stroke, a reperfusion injury, or a toxin-induced liver disease.

6. A method of inhibiting apoptosis in a cell, said method comprising transfecting said cell with nucleic acid encoding rHuAFP or an apoptosis-inhibiting fragment thereof.

7. The method of claim 6, wherein said cell is in a human patient.

8. The method of claim 7, wherein said human patient is infected with HIV, or has a neurodegenerative disease, a myelodyplastic syndrome, or an ischemic injury.


SEQUENCE LISTING (8 sequences)

SEQ ID NO: 1 - DNA, 2027 bp, Homo sapiens
atattgtgcttccaccactgccaataacaaaataactagcaaccatgaagtgggtggaatcaatttttttaattttcctactaaattttactgaatccagaacactgcatagaaatgaatatggaatagcttccatattggattcttaccaatgtactgcagagataagtttagctgacctggctaccatattttttgcccagtttgttcaagaagccacttacaaggaagtaagcaaaatggtgaaagatgcattgactgcaattgagaaacccactggagatgaacagtcttcagggtgtttagaaaaccagctacctgcctttctggaagaactttgccatgagaaagaaattttggagaagtacggacattcagactgctgcagccaaagtgaagagggaagacataactgttttcttgcacacaaaaagcccactgcagcatggatcccacttttccaagttccagaacctgtcacaagctgtgaagcatatgaagaagacagggagacattcatgaacaaattcatttatgagatagcaagaaggcatcccttcctgtatgcacctacaattcttctttcggctgctgggtatgagaaaataattccatcttgctgcaaagctgaaaatgcagttgaatgcttccaaacaaaggcagcaacagttacaaaagaattaagagaaagcagcttgttaaatcaacatgcatgtccagtaatgaaaaattttgggacccgaactttccaagccataactgttactaaactgagtcagaagtttaccaaagttaattttactgaaatccagaaactagtcctggatgtggcccatgtacatgagcactgttgcagagcagatgtgctggattgtctgcaggatggggaaaaaatcatgtcctacatatgttctcaacaagacactctgtcaaacaaaataacagaatgctgcaaactgaccacgctggaacgtggtcaatgtataattcatgcagaaaatgatgaaaaacctgaaggtctatctccaaatctaaacaggtttttaggagatagagattttaaccaattttcttcaggggaaaaaaatatcttcttggcaagttttgttcatgaatattcaagaagacatcctcagcttgctgtctcagtaattctaagagttgctaaaggataccaggagttattggagaagtgtttccagactgaaaaccctcttgaatgccaagataaaggagaagaagaattacagaaatacatccaggagagccaagcattggcaaagcgaagctgcggcctcttccagaaactaggagaatattacttacaaaatgagtttctcgttgcttacacaaagaaagccccccagctgacctcgtcggagctgatggccatcaccagaaaaatggcagccacagcagccacttgttgccaactcagtgaggacaaactattggcctgtggcgagggagcggctgacattattatcggacacttatgtatcagacatgaaatgactccagtaaaccctggtgttggccagtgctgcacttcttcatatgccaacaggaggccatgcttcagcagcttggtggtggatgaaacatatgtccctcctgcattctctgatgacaagttcattttccataaggatctgtgccaagctcagggtgtagcgctgcaaaggatgaagcaagagtttctcattaaccttgtgaagcaaaagccacaaataacagaggaacaacttgaggctctcattgcagatttctcaggcctgttggagaaatgctgccaaggccaggaacaggaagtctgctttgctgaagagggacaaaaactgatttcaaaaactggtgctgctttgggagtttaaattacttcaggggaagagaagacaaaacgagtctttcattcggtgtgaacttttctctttaattttaactgatttaacactttttgtgaattaatgaaatgataaagacttttatgtgagatttccttatcacagaaataaaatatctccaaa

SEQ ID NO: 2 - protein, 590 aa, Homo sapiens
TLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTAAWIPLFQVPEPVTSCEAYEEDRETFMNKFIYEIARRHPFLYAPTILLSAAGYEKIIPSCCKAENAVECFQTKAATVTKELRESSLLNQHACPVMKNFGTRTFQAITVTKLSQKFTKVNFTEIQKLVLDVAHVHEHCCRADVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNEFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQRMKQEFLINLVKQKPQITEEQLEALIADFSGLLEKCCQGQEQEVCFAEEGQKLISKTGAALGV

SEQ ID NO: 3 - protein, 197 aa, Homo sapiens
TLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTAAWIPLFQVPEPVTSCEAYEEDRETFMNKFIYEIARRHPFLYAPTILLSAAGYEKIIPSCCKAENAVECFQTKAATVTKELRES

SEQ ID NO: 4 - protein, 192 aa, Homo sapiens
SLLNQHACPVMKNFGTRTFQAITVTKLSQKFTKVNFTEIQKLVLDVAHVHEHCCRADVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQES

SEQ ID NO: 5 - protein, 201 aa, Homo sapiens
QALAKRSCGLFQKLGEYYLQNEFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQRMKQEFLINLVKQKPQITEEQLEALIADFSGLLEKCCQGQEQEVCFAEEGQKLISKTGAALGV

SEQ ID NO: 6 - protein, 389 aa, Homo sapiens
TLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTAAWIPLFQVPEPVTSCEAYEEDRETFMNKFIYEIARRHPFLYAPTILLSAAGYEKIIPSCCKAENAVECFQTKAATVTKELRESSLLNQHACPVMKNFGTRTFQAITVTKLSQKFTKVNFTEIQKLVLDVAHVHEHCCRADVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQES

SEQ ID NO: 7 - protein, 393 aa, Homo sapiens
SLLNQHACPVMKNFGTRTFQAITVTKLSQKFTKVNFTEIQKLVLDVAHVHEHCCRADVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNEFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQRMKQEFLINLVKQKPQITEEQLEALIADFSGLLEKCCQGQEQEVCFAEEGQKLISKTGAALGV

SEQ ID NO: 8 - protein, 325 aa, Homo sapiens
MSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNEFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQRMKQEFLINLVKQKPQITEEQLEALIADFSGLLEKCCQGQEQEVCFAEEGQKLISKTGAALGV